(19) United States
(12) Patent Application Publication
(10) Pub. No.: US 2016/0243334 A1
(43) Pub. Date: Aug. 25, 2016

(54) Self-limiting Optical Disinfecting Catheter

(71) Applicants: Luiz Barroca Da Silva, Livermore, CA (US); Donald M Cohen, Irvine, CA (US)

(72) Inventors: Luiz Barroca Da Silva, Livermore, CA (US); Donald M Cohen, Irvine, CA (US)

(21) Appl. No.: 14/628,290

(22) Filed: Feb. 22, 2015

(51) Int. Cl. A61M 25/01; A61M 25/00; A61M 5/44

(52) U.S. Cl. CPC A61M 25/0111; A61M 5/44; A61M 25/0017; A61M 25/0043; A61M 2205/36; A61M 2025/006; A61M 2209/10; A61M 2025/0019

(57) ABSTRACT

The present invention relates to an implanted catheter that reduces infection by heating a portion of the catheter wall. Light absorption within the wall is used to heat the surface. A thermochromic layer can be used to limit temperatures and accurately control the temperature distribution.

### BACKGROUND OF THE INVENTION

Each year in the United States, hospitals and clinics use more then 1 billion intravascular devices for the administration of intravenous (IV) fluids, medications, blood products, and parenteral nutrition fluids; to monitor hemodynamic status; and to provide hemodialysis. The majority of these devices are peripheral venous catheters, 15 million central venous catheters (CVC) are inserted each year. Bloodstream infections associated with CVCs are an important cause of morbidity and mortality in the intensive care unit. More than 16,000 episodes occur each year, with mortality estimates ranging from 3% to 25% (Mermel LA. Prevention of intravascular catheter-related infections. Ann Intern Med 2000; 132:391-402.). These infections are associated with increased lengths of stay and added hospital costs of up to $460 million per year in the United States alone. The risk factors for iv catheter-related infections vary according to the type of catheter; the hospital size, unit, or service; the location of the site of insertion; and the duration of catheter placement The pathogenesis of infection is often related to (1) extraluminal colonization of the catheter, which originates from the skin and, less commonly, from hematogenous seeding of the catheter tip, or (2) intraluminal colonization of the hub and lumen of the catheter. The microorganisms most commonly associated with peripheral vascular and CVC infection are coagulase-negative staphylococci, S. aureus, different species of aerobic gram-negative bacilli, and C. albicans.

The probability of infection can be reduced by strict adherence to sterile technique in the placement of catheters. Catheters that use ogliodynamic activity to kill bacteria have also been proposed by U.S. Pat. No. 5,409,467 by Raad et al. U.S. Pat. No. 5,037,395 by Spencer et al. describe the use of metal heaters to kill bacteria on urinary catheters using heat. Antimicrobial-coated catheters have also been recently developed that can reduce infection rates (Darouiche R O, Raad I I, Heard S O, et al. A comparison of two antimicrobial-impregnated central venous catheters. Catheter Study Group. N Engl J Med 1999; 340:1-8.) (and U.S. Pat. No. 6,626,873 B1 by Modak et al.).

A disinfection system has been disclosed in which light in the ultraviolet to blue wavelength range (240-450 nm) is directed at bacteria which would then be killed through photodynamic production of oxygen radicals (Arakane K, Ryu A, Hayashi C et al). Singlet oxygen (1 delta g) generation from coproporphyrin in Propionibacterium acnes on irradiation. Biochem Biophys Res Commun 1996; 223: 578-82; Barry L. Taylor, et al. Electron Acceptor Taxis and Blue Light Effect on Bacterial Chemotaxis, JOURNAL OF BACTERIOLOGY, November 1979, p. 567-573). It has also been shown that UVA and blue light can induce intracellular pH changes that can damage and ultimately kill bacteria (Futsaether C M, Kjeldstad B, Johnsson A. Intracellular pH changes induced in Propionibacterium acnes by UVA radiation and blue light. J Photochem Photobiol B 1995; 31: 125-31).

McCoy et al disclosed in patent US20090292357 A1 a system in which an anti-bacterial and/anti-viral effect is achieved by a sensitizer which produces highly reactive singlet oxygen ^{|}O₂.

It is known that elevated temperature can have a lethal effect on micro-organisms. Application of heat to achieve high temperature has been used to sterilize medical devices for many decades. While this is practical for sterilization of devices before they are inserted into patient contact, it is not as simple to achieve the selective destruction of foreign micro-organisms without harming the patient/host on an in-vivo device.

A need exists for a convenient and easy to use system for disinfecting intravascular catheters, urinary tract catheters, and other catheters and dwelling medical devices that avoids the use of antimicrobial coatings that can lead to drug resistant strains and avoids excessive damage to the patient. The present invention fulfills this need, and further provides related advantages.

An objective of the invention is to provide a catheter that has a portion that can be heated by a controller to deter or reverse the proliferation of micro-organisms. A further objective is to heat the catheter in a manner that is inherently self-limiting, and thus averts the hazard of inadvertent excessive heating. In particular it is an objective to avoid localized excessively hot spots.

Often optical heating systems are prone to overheating. Temperature coefficient often rises as temperature rises, so the rate of heating tends to increase rapidly. There is therefore a need for an optical heating system that is relatively immune to this positive feedback run-away heating condition.

### SUMMARY OF THE INVENTION

The invention is a system and method for disinfecting catheters. This and other objects will be apparent to those skilled in the art based on the teachings herein.

One embodiment of the present invention is a system comprising a control unit, a catheter and a coupler to connect the two and deliver light from the control unit to the catheter. In normal use the system is used when a catheter is first inserted and then a maximum of four times a day but more commonly once every one to three days. Disinfection is performed by connecting the control unit to the catheter and initiating a disinfection procedure. In one embodiment the disinfection procedure consists of transmitting light into the catheter hub and lumen to effectively illuminate all surfaces that are susceptible to bacterial growth. The control unit turns off the light source when adequate optical flux has reached all important surfaces. The flux will be in the range of 1 J/cm2 to 500 J/cm2 which is adequate to effectively kill bacteria. The time of the disinfection procedure can depend on the type of catheter. In one embodiment the type of catheter can be determined automatically by the control unit when the cable is connected to the keyed hub of the catheter.

The control unit may be connected directly to the coupling hub of the catheter or with a cable. The connection may be permanent, or alternatively be capable of separating the catheter from the control unit for convenience.

In an embodiment of the present invention, the light source used is a high intensity flash or laser source that can provide a high amount of optical energy in a short duration. In this embodiment the light is absorbed in a thin layer on the catheter surfaces that quickly heats the layer to a high temperature typically less than 250° C. and for most applications less than 100° C. In this embodiment the light pulse is a short duration (typically less than 10 seconds) to quickly heat the thin layer without significantly heating any surrounding tissue or fluid. The wavelength of the laser source is selected to have very low absorption in tissue to eliminate the risk of local tissue heating. For example the wavelength of the laser could be in the range 600-1000 nm. The wavelength is chosen to have substantially higher absorption within a portion of the catheter than in the local tissue.

Another aspect of the invention is a method of use of the system of the present invention in the killing of bacteria that can coat the surfaces of a catheter or a transdermal medical device and lead to infection.

Other objects and advantages of the present invention will become apparent from the following description and accompanying drawings.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows how a transcutaneously implanted venous catheter would be connected for disinfection

FIG. 2 shows a sectional view taken through the catheter where it enters the body.

FIG. 3 shows a sectional view taken through the catheter hub and connector.

FIG. 4 shows a thin absorbing layer integrated into the inside surface of the catheter wall.

FIG. 5a shows a thin absorbing layer integrated into the outside surface of the catheter wall.

FIG. 5b shows the catheter of FIG. 5a with an additional layer covering the thin absorbing layer.

FIG. 6 shows structures integrated into the catheter polymer that interact with light to change the interaction of light with the catheter wall.

FIG. 7 shows light rays moving from one medium and into a different medium with lower refractive index; and how light is reflected and refracted.

FIG. 8 shows a sectional view taken through the distal end of an indwelling catheter in which arrows representing light rays are shown to indicate light transmission in the catheter wall, a distal reflector is depicted and shown to reflect light back towards the proximal end, body fluid including blood cells are depicted outside the catheter and a colony of micro-organisms are depicted colonizing on a portion of the outer wall of the catheter. Light ray 120 is depicted refracting through and heating up the micro-organism colony.

FIG. 9a depicts a section view of a portion of a catheter in which light rays 110 are guided within the lumen of the catheter reflecting off and refracting into the wall of the catheter. It displays arcs 125 representing heat delivered to the catheter wall resulting from absorbed refracted light.

FIG. 9b depicts a section view of the catheter of FIG. 9a in which light rays 110 are guided within the lumen of the catheter reflecting off and refracting into the wall of the catheter. It displays arcs 125 representing heat delivered to the catheter wall resulting from absorbed refracted light. It also depicts a region 130 of the catheter wall that has become clear due to a local rise in temperature that triggers this change in the appearance. It further depicts a light ray 140 refracting through the region of the catheter wall that has become clear as it exceeded the thermochromic transition temperature (and no arcs in that clear region—indicating that there is not light absorption in this clear region of the catheter).

FIG. 10a depicts a section view of a portion of a catheter in which light rays 110 are guided within the walls of the catheter 50. Arc waves 125 in the figure represent heat delivered to the catheter wall as the rays travel and lose some energy through absorption in the wall.

FIG. 10b depicts the catheter of FIG. 10b in which a region 130 has become clear as a result of the temperature within that zone exceeding the thermochromic transition temperature. It further depicts (by the lack of heat waves 125 in this region) the diminution of light absorption in this clear region.

FIG. 11 depicts light rays 110 radiating outward from an optical fiber 140 within the lumen of the catheter 50.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

The present invention is a catheter disinfecting system that consists of a control unit, an in-dwelling catheter, and a coupler that connects the control unit to the catheter and delivers light to a portion of the catheter at sufficient flux and for sufficient time to heat micro-organisms to a lethal temperature.

The coupler may permanently or reversibly connect the catheter and controller. It may be rigid or flexible. It may couple the light directly from the light source to the catheter or indirectly as through fiber optic cable, lenses and/or reflectors.

While any portion of the catheter can be treated, there are locations that are more likely sites for microbial colonization, and thus are important targets for the disinfecting treatment. Such likely portions include the catheter entry site and the catheter hub.

When the control unit is connected to the catheter and the disinfecting processes starts light enters into the catheter hub and lumen and illuminates all the surfaces that could be coated with bacteria or fungus. The control unit determines the length of time for the process depending on the type of catheter and the desired total light flux on the areas to be disinfected. In most cases the total optical flux is less than 500 J/cm2 and for most applications less than 200 J/cm2. In one embodiment of the present invention, the control unit is always connected through an optical fiber to the catheter and light is continuously delivered at a low dose to prevent bacteria growth. In an alternative embodiment red light is used (˜650 nm) because it is highly scattered by hemoglobin in the blood and not well absorbed by the blood, thus inadvertent harm to the patient is diminished. The catheter material is chosen so that light is not strongly absorbed within the bulk of the catheter. The light causes substantial heating only where other material is present to cause the temperature elevation.

In an embodiment, of the present invention the light is absorbed in a thin layer on the catheter surfaces that quickly heats the layer to a high temperature typically less than 200° C. and for most applications less than 100° C. In this embodiment the light pulse is a short duration (typically less than 10 second) to quickly heat the thin layer without deleteriously heating any surrounding tissue or fluid. Bacteria are sensitive to high temperatures and can be killed effectively by a high temperature pulse. Preferably, the temperature achieved in the thin absorbing layer is greater than about 45° C. The preferable range of temperatures is from about 45° C. to about 200° C. In a preferred embodiment, the very high temperatures disclosed herein can be applied to the catheter surface without causing burns to the patient by limiting the exposure time. In addition to a single high temperature pulse a series of high temperature pulses can be produced to kill bacteria without risking any thermal damage or burn to tissue.

In a preferred embodiment, the controller and coupler are rugged and durable and suitable for use on multiple patients. The catheter is typically used on only a single patient. In order to prevent contamination of the reusable devices, a disposable single use interface connector device can functionally connect to the disposable single use catheter and serve as a barrier to contamination.

### System

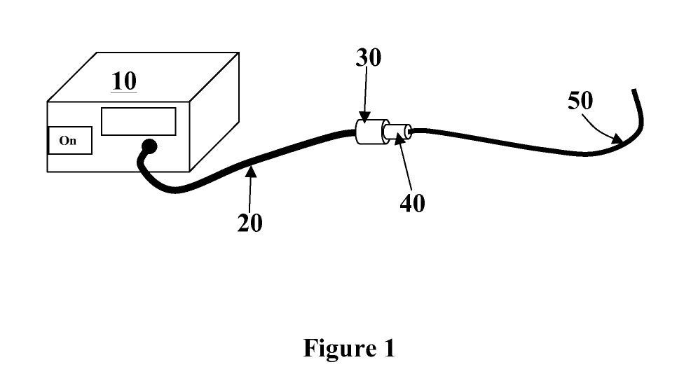

FIG. 1 shows the main components of the system. An electronic control unit 10 contains a light source and necessary control electronics. A cable 20 includes fiber optics to transport light and optional electrical wires that are used to detect the type of catheter or connector. A connector 30 at the end of the cable 20 connects to the catheter hub 40 which is part of the catheter 50. The connector 30 can be part of the cable 20 or a single use disposable connector that interfaces between the cable 20 and catheter hub 40. The function of the connector 30 is to provide good optical coupling between the fiber optics and the catheter 50.

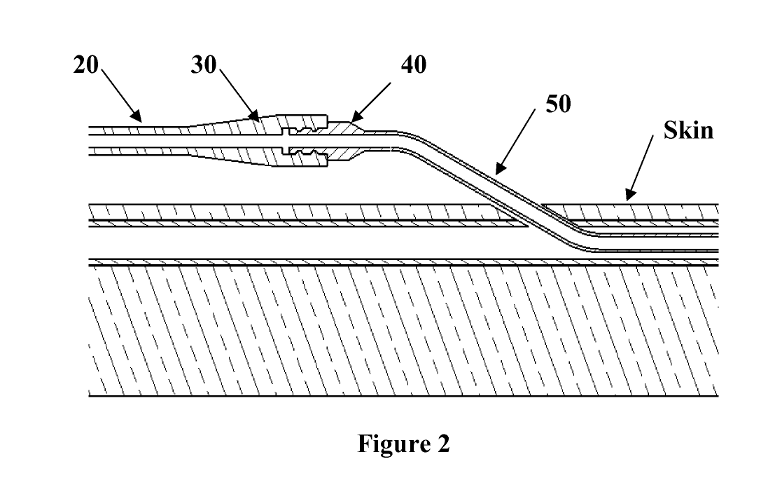

FIG. 2 shows how a transcutaneously inserted venous catheter 50 could be reversibly connected to the connector 30 before initiating the disinfection procedure. If necessary the IV tube is first disconnected from the catheter hub 40 and then the connector 30 couples to the catheter hub 40. In an alternative embodiment, the optical connection would be aligned with the long axis of the catheter, and the fluid connection would be through a side hole hub connection. This embodiment allows the fluid and optical connections to be simultaneously present.

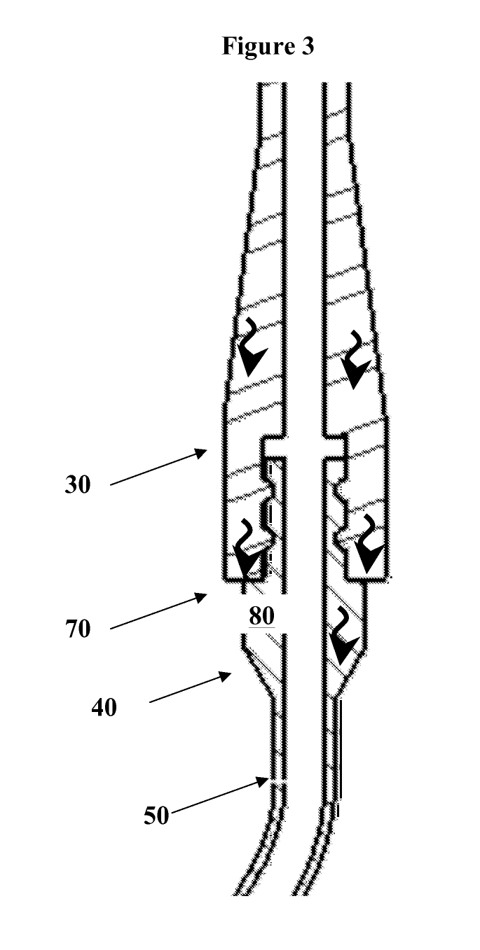

FIG. 3 shows a cross section through the catheter hub 40 and the connector 30. The connector 30 directs light at the catheter hub 40 to kill bacteria within the catheter hub 40. The connector 30 also couples light directly into the catheter 50 through the contact surface 70 and the optical taper section 80. The index of refraction of the optical taper 80 and catheter lumen 70 materials allows optical guiding of the light into the wall of the catheter 50 to directly illuminate all surfaces to be treated. The connector 30 is also designed to direct light at the catheter hub 40 to disinfect all the surfaces. Alternatively the light can be coupled directly to the catheter without a taper and without a fiber optic bundle.

Light could be coupled to the catheter by a fiber optic cable or other optical light guide, or by rigid optical elements. The light source may be a lamp such as an arc lamp, filament lamp or other equivalent. Alternatively it may be a laser, laser diode or light emitting diode.

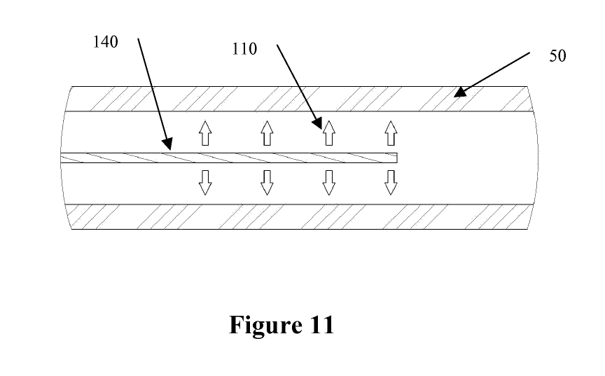

The light may be guided along the length of the catheter, or alternatively it may be directed at the walls of the catheter from an internal source such as an optical fiber or other functionally equivalent structure. An optical fiber for this purpose may direct the light from at or near its distal end essentially radially into the catheter. The light would preferably be directed nearly symmetrically radiating away from the long axis as depicted in FIG. 11.

In other alternatives the light may be directed from a lossy internal fiber from which light refracts out along the length. The intensity of the light could be uniform along the length and circumference, though in the preferred embodiment the light flux and intensity are highest where aligned with the catheter sites most susceptible to infection and microbial colonization, such as the entry site.

In yet other alternatives the light exposure is controlled by the rate of insertion or withdrawal of an optical fiber within the lumen of the catheter as light from the fiber impinges upon the catheter walls. Alternatively, the light exposure can be controlled by controlling the light emission at the source.

Light that is directed into the catheter interacts with the catheter and structures or materials incorporated into the catheter to cause a temperature rise that has a disinfecting effect on micro-organisms on the catheter.

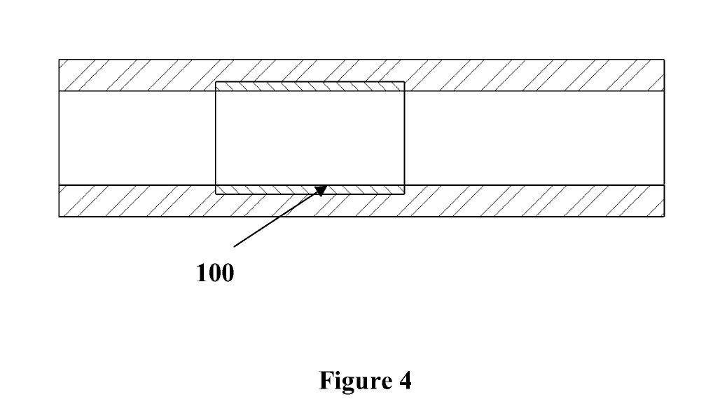

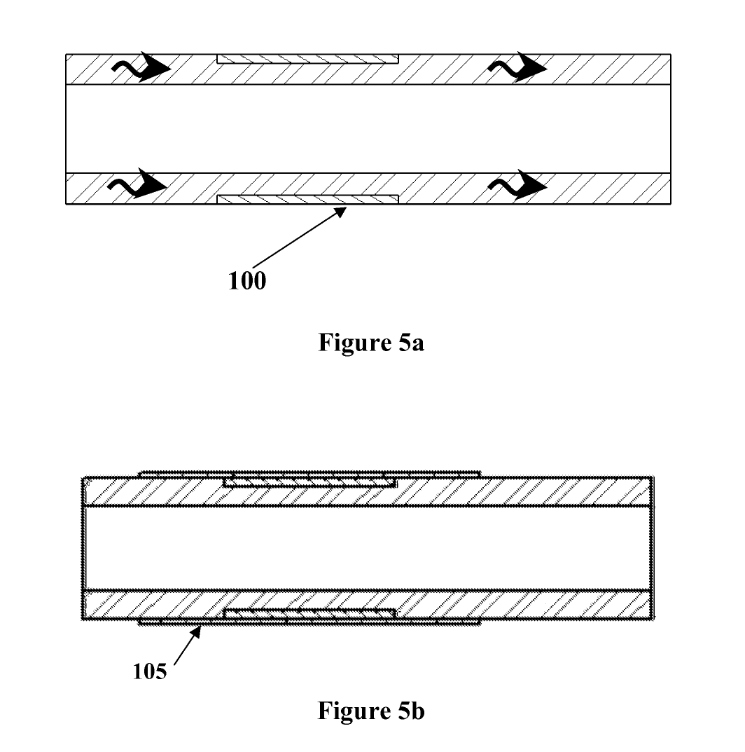

FIG. 4 and FIG. 5a and b show how high optical absorbing layers 100 could be integrated into sections of the catheter to achieve local heating and disinfection. The absorbing layers can be either on the inside surface or outside surface or both. The absorbing layers can be covered by another layer 105 as in FIG. 5b. The layer 105 can serve the purpose of improved bio-compatibility or improved lubricity. In one embodiment of the present invention all surfaces of the catheter 50 have high absorption to allow all surfaces to be heated. In another embodiment, only areas where bacterial growth is expected would have high absorption surfaces. Reducing the total area to be heated reduces the total optical energy required to disinfect the catheter. In another embodiment the high optical absorbing layers 100 are made of thermochromic polymers or include thermochromic pigments and colorants which undergo a reversible color change at the desired temperature (typically >45° C.). In this embodiment when the temperature of the absorbing layer is below the desired temperature the optical absorption is high to quickly heat the polymer. When the temperature exceeds the desired temperature the optical absorption drops significantly to reduce absorption and stop heating of the polymer. The advantage of this approach is that it eliminates the risk of very high local temperatures which could burn tissue. The other advantage is that it reduces the requirements on light uniformity over the complete area of the absorbing layer 100. Suitable thermochromic polymers can be found at http://www.thermochromic-polymers.com/index.html. For an example on thermochromic polymers refer to the paper (Reversible thermochromic effects in poly(phenylene vinylene)-based polymers, J. M. Leger, A. L. Holt, and S. A. Carter, Appl. Phys. Lett. 88, 111901(2006)) and references therein.

Thermochromic materials have temperature dependent absorption coefficients which change predictably and reversibly. For this application it is preferred that the transition temperature is above 40° C. Preferably the thermochromic material is chosen such that absorption coefficient is substantially higher when the material temperature is below the transition temperature than when it is above the transition temperature

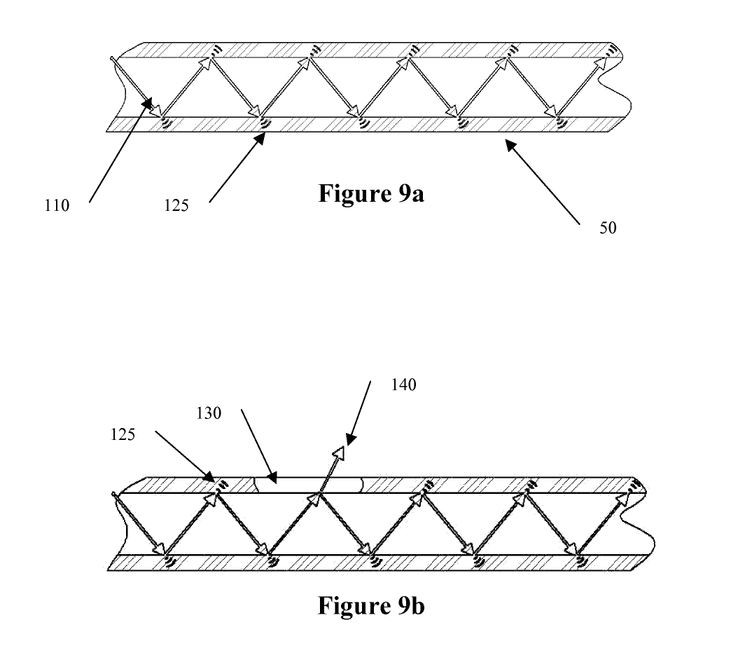

FIG. 9a depicts light rays 110 illuminating catheter 50 which in this case has been uniformly doped with thermochromic pigment. At every location where a light ray interacts with the pigment in the catheter, some light is absorbed and converted to heat as indicated by the arcs 125 and thus increases the temperature at that site.

The temperature at catheter location 130 is depicted to have exceeded the transition temperature of the thermochromic pigment, thus the catheter has become clear at location 130 as indicated by the absence of cross-hatching. Because the catheter is clear at this one location, light rays incident on this location transmit through (light ray 140) the clear catheter wall without being absorbed. Since catheter wall light absorption has ceased, so has heating. The temperature no longer rises. In this way, the system inherently prevents hot spots, and run-away overheating.

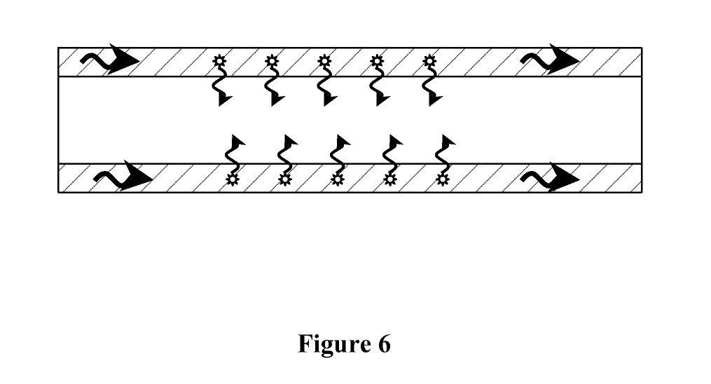

FIG. 6 shows how induced non-uniformities in the catheter wall can be used to increase the amount of light that is scattered or reflected towards the area to be treated or the absorbing layer to be heated. These non-uniformities can be produced by implanting plastics with different index of refraction or by using UV light to write patterns into the polymer. Alternatively the light can be directed towards the areas to be treated by particulate inclusions or air inclusions in the extruded catheter. The inclusions can be designed to cause scattering. A large portion of the scattered light would exceed the critical angle of the catheter light guide—and that light would be largely refracted out of the catheter rather than being guided within it. The scattering would thus redirect light towards the target. The distribution of the scatter inducing inclusions can be uniform or non-uniform. A logarithmically increasing degree of scattering inclusions along the catheter length could produce a nearly uniform light delivery along the walls of the catheter—as the magnitude of the light being guided will be expected to decline exponentially. A concentration of inclusions could be incorporated to intensify light delivery to those regions that require extra exposure. Alternatively inclusions can be selected for absorption rather than for scattering characteristics. Such inclusions would be placed superficially—near the inner or outer wall. These inclusions would heat by absorption of light radiation, then conduct heat towards the microbes.

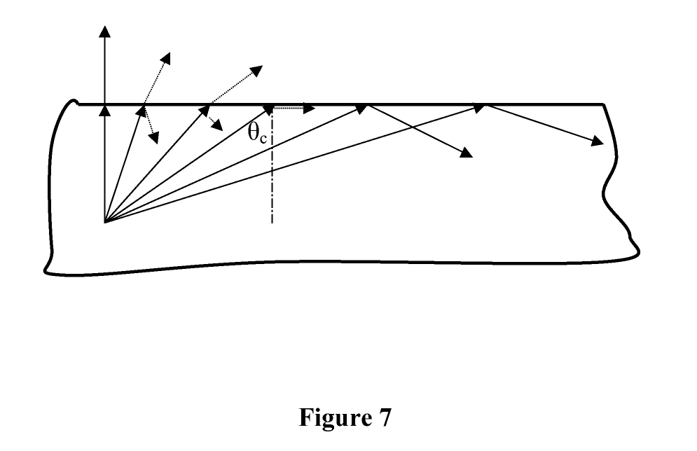

FIG. 7, shows how light is reflected and refracted when traveling towards a lower refractive index material. The angle of refraction can be calculated in conformance with Snell's Law n1 sin θ1=n2 sin θ2 It can be appreciated that there is a critical angle, θc , above which no light will refract into the less dense medium; rather it will stay confined within the higher refractive index material.

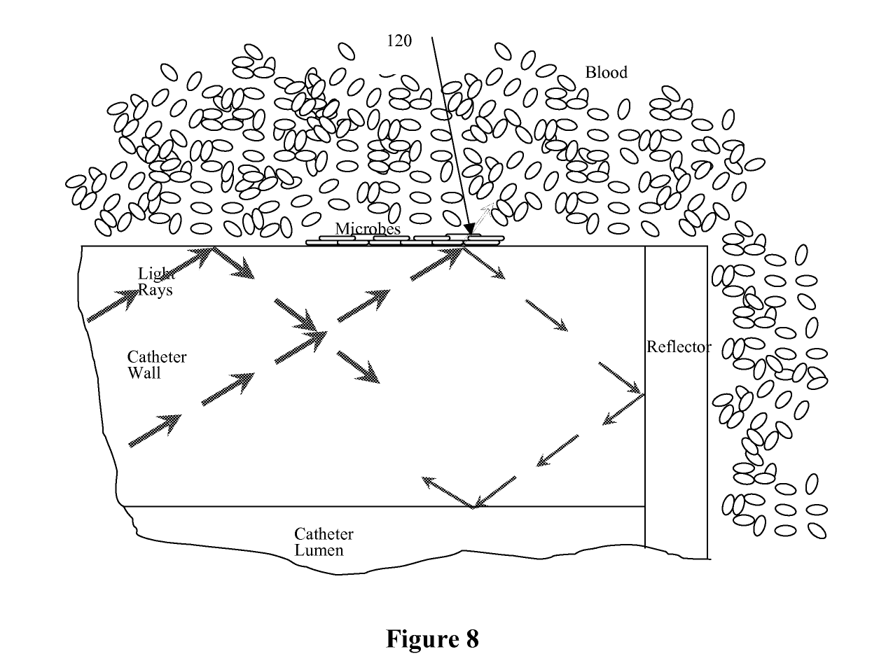

FIG. 8 shows the distal end of the catheter immersed in a biological medium such as whole blood. A colony of microorganisms is depicted on a surface of the catheter. Light rays are seen to be optically guided by the catheter. The angle made by these rays relative to the normal to the interface are generally greater than the critical angle. The critical angle is dependent on the environment in which the catheter is clad. The catheter will guide steeper rays in air than it will in blood. This is because the refractive index of blood is higher than that of air. Presumably the refractive index of blood is similar to that of water 1.33. The refractive index of lipid is approximately 1.45 according to the reference: http://stl.uml.edu/PubLib/Domankevitz,%20delivery.pdf

| | Cladding | Catheter | Critical |
|---|---|---|---|
| | Refractive | Refractive | Angle |
| | Index | Index | (degrees) |
| Air | 1 | 1.5 | 42 |
| Water | 1.33 | 1.5 | 62 |
| Lipid | 1.45 | 1.5 | 75 |

The foregoing table illustrates this effect. It further shows that when clad in lipid, the critical angle is even larger. Light rays steeper than 62° which were previously guided by the catheter would largely refract out of the catheter and into the lipid. Since microorganisms are bounded by cell membranes that are primarily composed of lipids, where there is a microbial colony on the catheter body, the catheter would be essentially lipid clad. In one embodiment of the invention, the light guided by the catheter contains substantial power in which the light rays are near the critical angle for blood (water). In this way, light irradiation is largely sequestered to those regions in which there is microbial colonization and substantial areas of intimate cell membrane contact. Such areas thus receive selective irradiation and heating—and are thus irradiated to a lethal level while the other regions are not.

FIG. 8 also shows that this embodiment of the catheter includes a reflector at the distal end. This allows the light to make 2 passes of the catheter—thereby reducing the power requirement of the light source to achieve the same flux in the tissue.

FIGS. 9a and 9b show a portion of a catheter 50 in section view. The catheter in this embodiment includes thermochromic material. The thermochromic material is blended in with the base polymer during the catheter extrusion manufacturing process. The catheter thus doped with the thermochromic pigment has a finite non-zero absorption coefficient for light in the waveband used.

The lumen of the catheter 50 in this embodiment is used as a light guide. Light ray 110 can be seen to impinge on the inner wall of the catheter. At each interface the light is partially reflected and a partially refracted. The portion that is refracted can interact with the thermochromic material within the catheter wall. This results in absorptive heating and temperature rise of the catheter wall where light is absorbed by the pigment as at location 125. As time passes, and more light is absorbed, the temperature continues to rise. Eventually the catheter wall achieves a desired temperature elevation for a desired time to have a desired inhibitory or lethal effect on microbial growth on the catheter.

As light exposure continues beyond this, the temperature may continue to rise. If allowed to progress unchecked, this could have an undesirable effect beyond just the beneficial disinfecting effect. The light wave band, intensity and exposure are selected so that the thermochromic material within the catheter wall achieves a self-limiting behavior that limits overheating. The light waveband is chosen so that a significant amount is absorbed by the thermochromic material when the temperature is below the transition temperature, and very little is absorbed above the transition temperature.

In the preferred embodiment, thermochromic pigment is extruded blended into the catheter in a concentration and particle size sufficient to allow rapid heating as a consequence of light absorption. Typically this will be less than 5% concentration. The thermochromic material may be procured as free flowing powder pigment available commercially from LCR Hallcrest for example.

As a hot spot develops perhaps due to some non-uniformity, the thermochromic material within the catheter wall exceeds a transition temperature. Above this transition temperature, the thermochromic pigment becomes clear as at hot spot 130; it greatly reduces its absorption of light. So even though light continues to impinge upon the catheter, the light absorption has been reduced, and thus the temperature rise slows. When this spot cools down below the transition temperature again, the thermochromic pigment changes from clear to visible again, indicative of the resumed absorptivity. Light incident on this site can again be absorbed by the thermochromic pigment, and again allow the catheter to heat for the purpose of disinfection. Again, wherever a hot spot develops, the thermochromic pigment behavior allows that spot of the catheter to become more clear, thus reducing heating. The heating process is thus self-limiting to reduce excursions and heating much above the transition temperature.

In the production process, the thermochromic doped layer may be covered by a co-extruded layer, interior and/or exterior. This may be desirable for enhance biocompatibility for example.

The light illuminating the catheter wall may emanate from an optical fiber within the catheter lumen, a illustrated in FIG. 11.

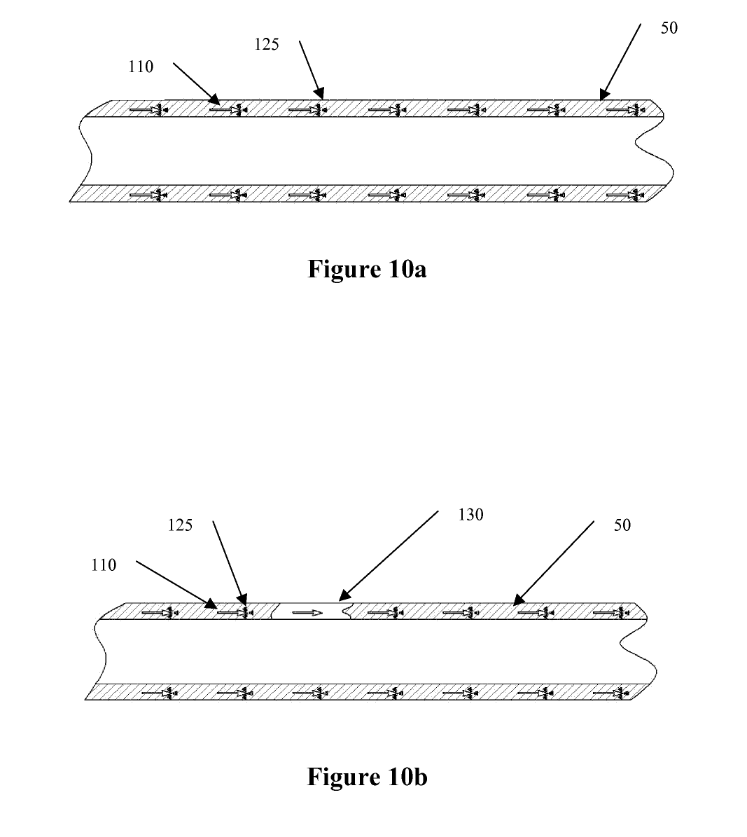

FIGS. 10a and b demonstrate the same principle, though in these figures the catheter wall itself is used as the light guide.

In an alternative embodiment a fiber optic cable is integrated into the catheter and is connected to the control unit immediately after the catheter is inserted. In this embodiment the light flux is less than 100 J/cm2 over the treatment area, and possibly less than 10 J/ cm2.

Thermochromic inks or dyes are temperature sensitive materials that reversibly change optical properties with exposure to heat and change in temperature. They may be liquid crystals or leuco dyes. Many materials exhibit small degrees of thermochromism; subtle change in color with temperature. Examples of materials with more pronounced thermochromism include: Cuprous mercury iodide, Silver mercury iodide, Mercury(II) iodide, Bis(dimethylammonium) tetrachloronickelate, Bis(diethylammonium) tetrachlorocuprate, Nickel sulfate, Chromium(III) oxide:aluminium(III) oxide and Vanadium dioxide

The design and structure of the catheter/controller system will mitigate hazards; for example the controller can limit the amount of energy delivered to the catheter. It can even limit the amount of energy delivered to particular subsections of the catheter, assuring that the average temperature in a given region is within desired limits. There is still a chance however that local regions could become overheated, for example due to variability in the construction or due to a kink in the catheter. There is thus a desire to incorporate technology that creates limits to over-heating such as the self-limiting heating of the thermochromic catheter.

There are many ways to achieve a self-limiting heating catheter. For an optical heating catheter, one way is to employ a thermochromic dye with appropriate transition temperature and scattering, absorption, reflection and transmission properties. The dye is applied to the region of the catheter to be heated. The controller allows delivery of light to the region. The dye absorbs enough of the light to heat the catheter and any proximate micro-organisms appropriately. In the event any segment gets even hotter—as could be caused by a kink in the catheter—the thermochromic dye color changes so that absorption is reduced. Overheating is thus self-limiting.

Many variations could be made in the optical system to achieve the desired result. An optical transition that reflects the light away from the overheating region can be effective. Alternatively, an optical transition that transmits the light instead of absorbing it can also be effective.

Though the preferred heating energy source has been described as optical, other energy sources could be employed in the self-limiting manner of the present invention. In other alternative embodiment, the heating is accomplished with apparatuses other than optical, for example ohmic heating, ultrasonic heating or other heating Ohmic heating can be accomplished by using a heater element of metal, conductive polymer, carbon, silicon, doped semiconductor or other suitable material.

There are other ways to heat the catheter other optically. Accordingly there are other ways to create the self-limiting feature to prevent local overheating. For an electrical heating catheter, the heating element can be constructed in a manner consistent with an electrical fuse, so that over-heating causes the over-heated section of the heating element to burn out without halting heating at the other locations. Alternatively, the self-limiting electrically heating catheter can used a heating element with a negative temperature coefficient of electrical resistivity. Most metal electrical conductors have positive temperature coefficient of electrical resistivity, i.e. the resistances rises if temperature rises. For such a material operated at constant voltage, a local hot spot could cause positive feedback and get even hotter; because it is hot, the resistance rises; that increases the temperature even further, and so on. A heating element with negative temperature coefficient of electrical resistivity would be self-limiting. A hot spot would cause the local electrical resistance to decline and thus limit the over-heating. Examples of materials with negative coefficient include carbon, silicon and germanium.

The heating can be localized at a location that is most susceptible to infection and microbial growth. For example, a heating element can be situated on the outer surface of the catheter encompassing and proximate the region where the catheter traverses from extra-corporeal to intra-corporeal.

### Method of Use

The devices described herein are suitable for use in the treatment and control of catheter based bacterial, viral or fungal infections.

### Pulsed Mode of Use of Disinfecting Catheter System:

Once a week or more frequently the catheter is disinfected as follows.
Catheter Hub is disconnected from external IV if necessary
Control Unit cable is connected to catheter hub
Control Unit begins disinfection process by activating light and exposing all surfaces for the time required to kill greater than 90% of the bacteria.
Control Unit cable is disconnected from catheter hub.
   Pulsed Mode of Use of Disinfecting Catheter System with Integrated Light Input and Hub:

Once a week or more frequently the catheter is disinfected as follows.
Control Unit is activated and a disinfecting cycle initiated.
Light propagates through the cable and into the catheter Hub and catheter.
Light stays on for the necessary time and then turns off.

### Continuous Mode of Use of Disinfecting Catheter System:

Immediately after the catheter is inserted, the fiber optic cable is connected to the control unit. The control unit is powered on and light is continuously transmitted into the catheter hub and wall.

The above descriptions and illustrations are only by way of example and are not to be taken as limiting the invention in any manner One skilled in the art can substitute known equivalents for the structures and means described. The full scope and definition of the invention, therefore, is set forth in the following claims.

What is claimed is:

1. A disinfecting catheter system apparatus comprising:
a control unit with a light source that emits a light;
a catheter with a wall;
a coupler to couple a portion of the light into the catheter wall;
wherein material is incorporated into a portion of the catheter wall to interact with the light to cause heating of the catheter wall.

2. The apparatus of claim Error! Reference source not found. in which the material is chosen for an optical property chosen from the list containing: high scattering and high absorption.

3. The system of claim 1 in which the material is a thermochromic material.

4. The system of claim 3 in which the thermochromic material has a transition temperature of at least 40° C.

5. The system of claim 3 in which the thermochromic material has a transition temperature and a reversible temperature dependent absorption coefficient, wherein the absorption coefficient is substantially higher below the transition temperature than it is above the transition temperature.

6. The system of claim 1 in which the light is guided by an optical fiber.

7. The system of claim 1 in which the light is guided through the body of the catheter.

8. The system of claim 1 in which the light is of sufficient intensity and wavelength to cause a lethal change in microbial growth on the catheter surface.

9. The system of claim 1 in which a flux of the light through the catheter wall is at least 1 Joule/cm².

10. The system of claim 1 in which the catheter wall is subject to an adherent layer of microbial growth;
wherein a portion of the light is transmitted into the adherent layer;
and the portion transmitted into the adherent layer is sufficient to cause an increase in temperature in the layer to at least 45° C.

11. The system of claim 1 in which the light includes radiation in a waveband; wherein the waveband is 600 nm to 1000 nm.

12. The system of claim 2 further including a structure within the catheter wall to scatter at least a portion of the light wherein the scattering structure is chosen from the list: air inclusions, particulate inclusions, etched wells, dye, ink and thermochromic material.

13. The system of claim 1 wherein the heating causes a rise in temperature to at least 45° C.

14. The system of claim 1 wherein the heating results in a temperature to a level within the range of 40° C. to 60° C.

15. The apparatus of claim 1 wherein the temperature of a portion of the catheter is heated to a level within the range of 40° C. to 150° C.

16. The apparatus of claim 1 wherein the light has a principal axis; wherein the principal axis is aligned with the long axis of the catheter, wherein the catheter includes at least one lumen for communication of a fluid, wherein access to the channel is through a port made through a side wall of the catheter.

17. The apparatus of claim 1 wherein the catheter includes a reflector at the distal end of the catheter.

18. The apparatus of claim 7 wherein at least a portion of the catheter body that guides light is unclad, where microbes are apt to colonize on the unclad portion;
wherein the optical property including refractive index of the catheter body is chosen so that a portion of the light directed into the catheter body will refract into the microbial colony;
wherein the refracted light is sufficient to cause a temperature rise in the microbial colony.

19. A self-limiting thermal disinfecting catheter system apparatus comprising:
a catheter with a wall;
a control unit with an energy source that transfers energy into the catheter wall; wherein the heating is self-limiting in a local region without halting heating at other locations.

20. A method for reducing catheter borne infections comprising applying light to heat a catheter wall sufficient to cause lethal damage to micro-organisms on the catheter wall, in which the catheter wall has structure that interacts with the light to limit excessive local heating.
